(19) United States
(12) Patent Application Publication
(10) Pub. No.: US 2023/0284964 A1
(43) Pub. Date: Sep. 14, 2023

(54) METHOD AND DEVICE FOR THE OBJECTIVE CHARACTERIZATION OF SYMPTOMS OF PARKINSON'S DISEASE

(71) Applicant: Brain Innovations, S.r.l., Roma (IT)

(72) Inventor: Lazzaro DI BIASE, Roma (IT)

(21) Appl. No.: 18/025,069

(22) PCT Filed: May 25, 2021
(86) PCT No.: PCT/IT2021/050160

§ 371 Date: Mar. 7, 2023

(30) Foreign Application Priority Data
Sep. 10, 2020 (IT) 102020000021382

(51) Int. Cl. A61B 5/00; A61B 5/11

(52) U.S. Cl. CPC A61B 5/4082; A61B 5/1101; A61B 5/7264; A61B 5/681

(57) ABSTRACT

A method and a system are described for performing an objective characterization of symptoms of the Parkinson's disease, through an analysis of the motion status of patients, wherein said motion status can be in one of the following phases: OFF phase, wherein Parkinson's symptoms, such as rigidity, tremor and bradykinesia, emerge; ON phase wherein the symptoms markedly improve; DIS phase wherein involuntary movements emerge, called dyskinesias; as detectors of the symptoms the following are used: voice analysis, face analysis, tremor analysis and movement analysis of said patient, an algorithm being provided adapted to use the detecting parameters to determine the motion status of the patient.

The present invention refers to a method, and to a device for the implementation of said method, for the objective characterization of the symptoms of Parkinson's disease.

The invention is part of the technical field of medicine, in particular neurology.

Parkinson's disease is the most frequent neurodegenerative disease after Alzheimer's disease. The diagnosis of Parkinson's disease is essentially clinical and currently based on the objective and anamnestic identification of the characteristic signs and symptoms of the disease and on the exclusion of any atypical symptoms.

There are specific diagnostic criteria, developed over the years, in order to standardize and systematize the diagnosis of Parkinson's disease.

These methods objectively characterize the symptoms of Parkinson's disease, but do not integrate movement, tremor, facial and vocal analysis data and, above all, do not provide real-time data to monitor the progress of the disease moment by moment.

Patients with Parkinson's disease can experience fluctuations in their motor status during the day going from an OFF phase, in which parkinsonian symptoms such as rigidity, tremor and bradykinesia emerge, to an ON phase in which these symptoms improve significantly. marked, and also to a phase characterized by involuntary movements called dyskinesias (DIS).

Object of the present invention is providing a method and a system, respectively in accordance with claims 1 and 2, suitable for characterizing the motor state of the patient.

The method for the objective characterization of the symptoms of Parkinson's disease, called characterization taking place through the analysis of the motor state of patients with said Parkinson's disease, in which said motor state may be in one of the following phases:
OFF phase, in which parkinsonian symptoms such as rigidity, tremor and bradykinesia emerge;
ON phase, in which said symptoms improve markedly;
DIS phase, in which involuntary movements defined as dyskinesias emerge;

is characterized by using voice analysis, facial analysis, tremor analysis and movement analysis of said patient as parameters for detecting said symptoms, an algorithm being provided for using said detection parameters to determine the motor state of said patient.

The system for the objective characterization of the symptoms of Parkinson's disease includes:
an electronic bracelet, equipped with an accelerometer, a magnetometer and a triaxial gyroscope, used to collect continuous data on tremor and movement;
a camera to capture facial expressions, to be activated at the request of the patient;
a recorder to record the patient's voice to be activated at the patient's request;
a processing device, equipped with suitable software which, using a special algorithm, integrates movement, tremor, facial and vocal data to identify the motor state of the patient with Parkinson's disease (ON, OFF, DIS);
a display showing the patient's motor status (ON, OFF, DIS).

According to a preferred embodiment, said camera, said recorder, said processing device, said display and the relative software are integrated in a palmtop/smartphone, whereby the electronic bracelet sends the combined data of movement and tremor analysis directly to said palmtop/smartphone, said palmtop/smartphone being provided with a display able to visualize the motor state of the patient: ON, OFF, DIS.

Preferred embodiments and non-trivial variants of the present invention form the subject of the dependent claims.

The advantage deriving from the use of the invention consists in the fact that it guarantees continuous monitoring 24 hours a day, integrating kinematics data with facial and voice analysis data.

It will be immediately obvious that innumerable variations and modifications (for example relating to shape, dimensions, arrangements and parts with equivalent functionality) can be made to what is described, without departing from the scope of the invention, as appears from the attached claims.

It is understood that all attached claims form an integral part of the present description.

The present invention will be better described by a preferred embodiment, given by way of non-limiting example, with reference to the attached drawings, in which:

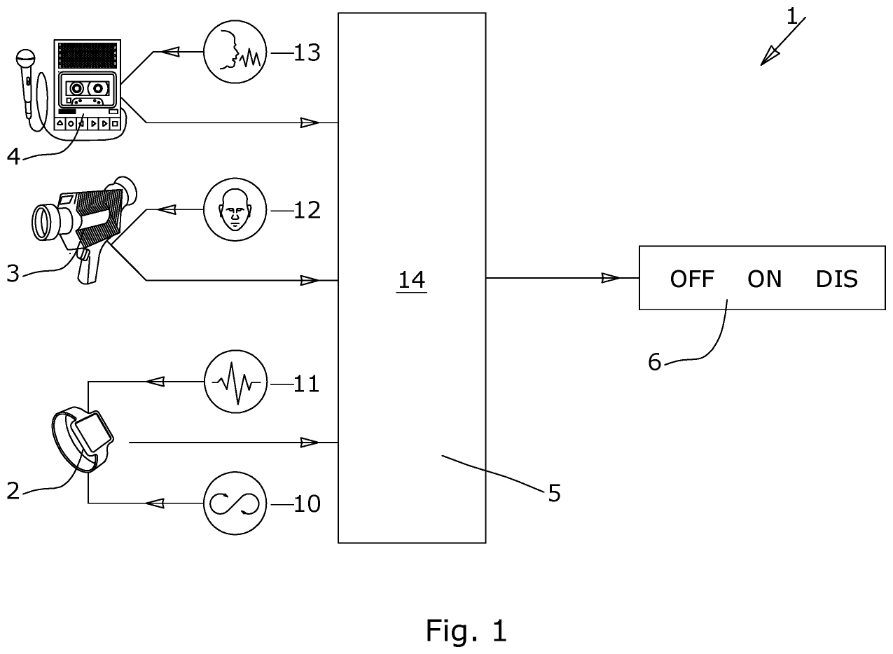

FIG. 1 shows a system for the objective characterization of the symptoms of Parkinson's disease, according to the invention;

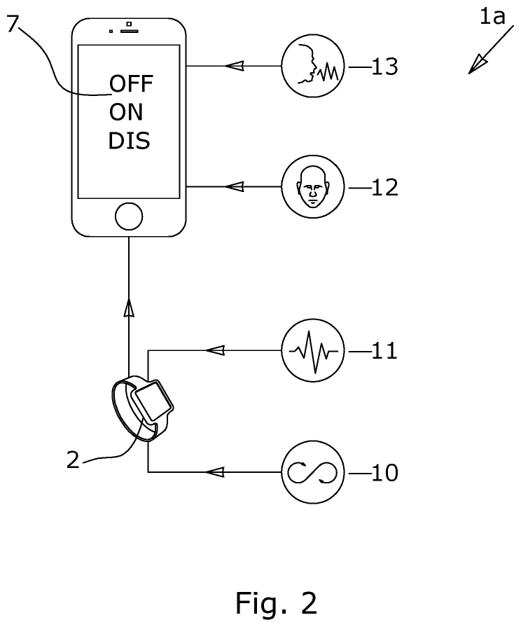

FIG. 2 shows a variant of the system according to the invention.

With reference to FIG. 1, the system (1) for the objective characterization of symptoms of Parkinson's disease includes:
an electronic bracelet (2), equipped with an accelerometer, a magnetometer and a triaxial gyroscope, used to continuously collect data on tremor and movement;
a camera (3) for recording facial expressions, to be activated at the request of the patient;
a recorder (4) to record the voice to be activated at the request of the patient;
a processing device (5), equipped with suitable software which, using a special algorithm, integrates movement, tremor, facial and vocal data to identify the motor state of the patient suffering from Parkinson's disease (ON, OFF, DIS);
a display (6) showing the patient's motor status (ON, OFF, DIS).

This system is used for the objective characterization of the symptoms of Parkinson's disease.

The operation of the system (1) is divided into the following steps.

Step 1—The electronic bracelet (2) continuously records the patient's movement (10) and tremor (11) data.

Step 2—When the patient perceives a state of OFF or marked dyskinesias (DIS), he uses the camera (3) to capture facial expressions and the recorder (4) to record his voice.

Step 3—The processing device (5) receives from the electronic bracelet (2), the camera (3) and the recorder (4) the combined data of movement (10), tremor (11), facial images (12) and item (13) and, through the motor state characterization algorithm, identifies the patient's motor state (14) and displays it on the display (6): ON, OFF, DIS.

According to a preferred embodiment (la) shown in FIG. 2, the camera (3), the recorder (4), the processing device (5), the display (6) and the related software are integrated in a PDA/smartphone (7), for which the electronic bracelet (2) sends the combined analysis data of movement (10) and tremor (11) directly to the PDA/smartphone (7), which carries out the analysis by applying the motor status characterization algorithm indicates on its display the patient's motor status: ON, OFF, DIS.

1. A method for performing an objective characterization of symptoms of Parkinson's disease, comprising determining a motor status of a patient affected by said Parkinson's disease, wherein said motor status can be in one of the following phases:
OFF phase, wherein Parkinson's symptoms, comprising one or more of rigidity, tremor and bradykinesia, emerge;
ON phase, wherein said symptoms markedly improve;
DYS phase, wherein involuntary movements emerge, called dyskinesias;
wherein said method uses as detectors of said symptoms: voice analysis, face analysis, tremor analysis and movement analysis of said patient, wherein an algorithm is provided that is adapted to use said detecting parameters to determine the motor status of said patient.

2. A system for the objective characterization of symptoms of Parkinson's disease, comprising:
an electronic bracelet, equipped with an accelerometer, a magnetometer and a gyroscope, three-axial, for collecting data continuously about tremor and movement;
a photo-camera to shoot face expressions, to be actuated upon request of a patient;
a recorder to record the patient voice, to be actuated upon request of the patient;
a processing device, equipped with a suitable software which, using a suitable algorithm, integrates data about movement, tremor, face and voice to identify a motor status of the patient affected by Parkinson's disease, comprising ON, OFF, and DYS;
a display on which the motor status of the patient is displayed.

3. The system for the objective characterization of symptoms of Parkinson's disease according to claim 2, wherein said photo-camera, said recorder, said processing device, said display and the related software are integrated in a handheld computer/smartphone, so that the electronic bracelet sends combined data about analysis of movement and tremor directly to said handheld computer/smartphone, said handheld computer/smartphone being equipped with a display adapted to display the motor motion status of the patient: ON, OFF, DYS.
